(19) United States
(12) Patent Application Publication
(10) Pub. No.: US 2021/0246272 A1
(43) Pub. Date: Aug. 12, 2021

(54) LOOSE WAX POWDER AND PROCESS FOR MANUFACTURING SUCH A POWDER

(71) Applicant: Arkema France, Colombes (FR)

(72) Inventors: Guillaume LE, Serquigny (FR); Helene EGRET, Colombes Cedex (FR); Helena CHEMINET, Wauwatosa, WI (US); Geoffroy Cammage, Serquigny (FR); Amelie NOEL, Serquigny (FR)

(21) Appl. No.: 17/049,158

(22) PCT Filed: Apr. 19, 2019
(86) PCT No.: PCT/FR2019/050952

§ 371 Date: Oct. 20, 2020

(30) Foreign Application Priority Data
Apr. 25, 2018 (FR) 1853635

(51) Int. Cl. C08J 3/12; A61K 8/02; B29B 9/10; A61K 8/92; B29B 9/12

(52) U.S. Cl. CPC C08J 3/122; A61K 8/025; B29B 9/10; A61K 8/022; A61K 8/922; B29B 2009/125; A61K 2800/10; A61K 2800/412; A61K 2800/805; C08J 2391/06; B29B 9/12

(57) ABSTRACT

The present invention relates to a loose wax powder obtained from renewable materials, in which the particles have a particle size such that:
the D90v is less than 20 microns
the SPAN calculated by the formula (D90-D10)/D50 is less than 1.8
the volume percentage of particles with a diameter of at least 30 microns is less than 5%. The present invention also relates to a process for preparing such a powder.

### FIELD OF THE INVENTION

The present invention relates to fine powders, such as those used in cosmetics, pharmaceuticals or perfumery. The present invention relates more particularly to a fine wax powder obtained from renewable materials. A subject of the invention is also a process for manufacturing such a powder obtained from renewable raw materials.

The ingredients found in conventional cosmetics are essentially of petroleum origin or of synthetic origin. The processes for obtaining them are occasionally considered to be environmentally polluting.

Specifically, the raw materials used for the synthesis of these ingredients are obtained by steam cracking or catalytic cracking of petroleum cuts. The use of these materials contributes toward the increase in the greenhouse effect. Given the worldwide reduction in oil reserves, the source of these raw materials will gradually diminish.

The cosmetics market is evolving under the impetus of new regulations regarding the problem of “microplastics”: the pollution of waters with plastic particles which concentrate pollutants, which then become incorporated into the food chain. In the United States, the government decided to introduce a law (Microbead-Free Waters Act of 2015) prohibiting the use of petroleum-based polymers in certain body care products. There is thus a great demand from the cosmetics market for solutions for sensory powders which would not be affected by this problem.

Raw materials derived from biomass are of renewable source and have a reduced environmental impact. They do not require all the refining steps (which are very energy-intensive) of petroleum-based products. The production of CO2 is reduced, and as such they contribute less toward global warming.

It is thus apparent that it is necessary to have synthetic processes that are not dependent on raw material of fossil origin, but rather which use raw materials of renewable origin.

Nowadays, consumers are increasingly drawn toward products of plant origin which have the reputation of being safer and more skin-friendly.

Moreover, in a market which is as competitive as the cosmetics market, formulators must meet consumer demand for cosmetics which combine efficacy, an innovative texture and sensory qualities. Now, these properties depend not only on the raw materials but also on the processes used.

The aim of the present invention is thus to provide a fine powder, which meets the various requirements mentioned above, both in terms of efficacy, texture and sensory qualities, and in terms of ecological and biological ethics.

For the purposes of the invention, the term “fine powder” means a powder with a volume-median particle diameter (D50 or D50v hereinbelow) of less than 30 microns, preferably less than 20 microns, preferably in the range from 2 to 20 microns, preferably from 5 to 18 microns, preferably from 7 to 15 microns, preferably from 10 to 15 microns, according to the standard ISO 13319:2017.

The term “porous particles” denotes particles including pores. The porosity is characterized quantitatively by the specific surface area (also known as the SSA). The porous particles of the invention have an SSA measured according to the BET method of greater than or equal to 8 m2/g. The BET (Brunauer-Emmett-Teller) method is a method that is well known to those skilled in the art. It is notably described in the Journal of the American Chemical Society, volume 60, page 309, February 1938, and corresponds to the international standard ISO 5794/1 (appendix D). The specific surface area measured according to the BET method corresponds to the total specific surface area, i.e. it includes the area formed by the pores.

The term “loose powder particles” denotes particles that are not grouped in the form of an aggregate or an agglomerate.

The term “particles with a smooth surface” denotes particles whose surface is free of roughness.

### PRIOR ART

As examples of powders commonly used in cosmetics, mention may be made of those of mineral origin (talc, silica, kaolin, sericite, calcium carbonate or magnesium carbonate); oxides (TiO2, ZnO); those of plant origin (starch); those of animal origin (silk powder); and those of synthetic origin: polymethyl methacrylate (PMMA), nylon-12 or polyamide 12 (PA 12).

The powders of mineral origin have sensory properties inferior to those of synthetic powders. They often have a rough, dry feel. They may dry out the skin and give rise to irritation.

Polyamide 12 powders, although of petroleum origin, are particularly appreciated in cosmetics for the soft and silky characteristic that they give to formulations. PA 12 is found not only in makeup formulations (eyeshadows, foundations, lipsticks, mascaras, etc.) but also in care formulations (day creams, night creams, body milks), antisun product formulations, etc. To obtain the sensory properties required for these formulations, the powders must have a mean particle size of less than 100 μm, preferably less than 50 μm and more preferably less than 20 μm.

As examples of PA 12 powders for cosmetic formulations, mention may be made of the following commercial products: ORGASOL 2002 EXD NAT COS (Arkema), Nylonpoly WL 10 (Création Couleurs), Covabead N12-10 (LCW), SP500 (Toray), Ubesta (UBE), Tegolon 12-20 (Evonik).

Various manufacturing processes allow the production of polyamide 12 powders, and each lead to different powder characteristics.

Examples that may be mentioned include direct synthetic processes, which lead to polyamide 12 powders by polymerization of lauryllactam or of aminododecanoic acid. Depending on the type of process, it is possible to obtain perfectly spherical nonporous powders or spheroidal porous powders. In the latter case, mention may be made of the PA 12 powders sold by Arkema France under the name Orgasol®.

Dissolution/precipitation processes also exist, which result in polymer powders by dissolution of a polymer in a solvent, followed by reprecipitation in powder form. This type of process leads to spheroidal powders, of variable porosity.

For example, patent application DE4421454 describes a process for synthesizing polyamide 12 powder by dissolution and precipitation, the aim of the invention in said application being to obtain a PA 12 powder of spherical form and narrow particle size. The process used requires the use of a polyamide whose molecular mass and viscosity are high enough to permit its precipitation. Also, the powder obtained after precipitation has a diameter such that milling under harsh conditions is necessary to obtain a fine powder with a diameter of less than 30 micron.

However, in all the cases mentioned previously, the lauryllactam and the aminododecanoic acid polymerized to obtain these PA 12 powders are derived from petrochemistry. Furthermore, to obtain powders with a particle size of less than 20 microns, these processes use large amounts of solvents, which are themselves derived from petrochemistry.

Unlike polyamide 12 powders, polyamide 11 (PA 11) powders are manufactured from raw materials of plant origin.

Plant materials have the advantage of being able to be grown in large amount, according to demand, throughout the majority of the globe, and of being renewable. A renewable raw material is a natural, animal or plant resource, the stock of which can be reconstituted over a short period on the human timescale. It is in particular necessary for this stock to be able to be renewed as rapidly as it is consumed.

PA 11 is produced by the company Arkema. The raw material for PA 11 is castor oil, extracted from the plant of the same name (Ricin commun), from castor oil plant seeds. It is a robust, inedible crop which therefore does not compete with subsistence crops and which grows mainly in impoverished soils in semiarid regions. For the cosmetics market, Orgasol® Green Touch is a 100% plant-based, ultrafine PA 11 powder, with a volume-median diameter substantially equal to 10 micron, designed to offer both the unique esthetic properties of nylon powders and the advantages of a renewable raw material. This product offers a creamy, velvety feel and improved compaction. The environmental performance of polyamide 11 was evaluated via the life cycle analysis process, according to the standards ISO 14040-14044. The manufacturing process presents a yield that is optimized in each step to reduce the production of waste (E-factor=0.1%) and allows a real reduction in greenhouse gas emissions relative to a polymer of fossil origin (−2.7 tonnes of CO2 per tonne of polymer (CO2 footprint=−38%).

Finally, to address the problem of “microplastics” and the new demand for natural raw materials which respect the principles of green chemistry, a new standard ISO 16128 is directed toward classifying raw materials and cosmetic products according to their naturality index, taking into account both their origin and the process for producing them.

One aim of the present invention is thus to design a novel powder that is not only of renewable origin and of high performance in terms of sensory properties, but also biodegradable in water according to the standard OCDE301, and for which the production process does not require the intervention of laborious, energy-intensive or pollutant chemical or technological operations.

The aim of the present invention is also to provide a process for manufacturing fine powder which is simple, quick (involving the fewest possible steps) and easy to perform.

On the strength of its expertise in the manufacture of high-performance biosourced powders, the Applicant Company has now developed novel fine sensory powders based on a nonpolymeric but biodegradable substance, for which the favored, but not exclusive, fields of use are cosmetics, pharmaceuticals and perfumery. The Applicant has also found a process for manufacturing such powder particles by “spray-cooling” the premelted substance.

### DETAILED DESCRIPTION

More precisely, one subject of the present invention is thus a loose wax powder obtained from renewable materials, in which the particles have a particle size such that:
the D90v is less than 20 micron
the SPAN calculated by the formula (D90-D10)/D50 is less than 1.8
the volume percentage of particles with a diameter of at least 30 microns is less than 5%.

Advantageously, the volume-median diameter D50v is within the range from 2 to 20 microns, preferably from 5 to 18 microns, preferably from 7 to 15 microns, preferably from 10 to 15 microns.

Advantageously, the SPAN is less than 1.5, preferably less than or equal to 1.2.

Advantageously, the content of particles with a diameter of at least 30 microns is less than 3%, preferably less than 2%, preferably less than 1%.

Advantageously, the powder particles according to the invention are of substantially spherical shape, and preferably of spherical shape.

Advantageously, said particles have a sphericity index Is within the range from 1 to 1.2, preferably from 1 to 1.1, preferably substantially equal to 1, the Is being measured by the ratio between the largest diameter of the particle to the smallest diameter of the particle, the diameters being measured by SEM.

Advantageously, the wax particles have a smooth surface. In particular, the surface of the particles of the invention is free of roughness such as that which may be observed in the case of milled particles.

Advantageously, the wax is chosen from natural hydrocarbon-based waxes comprising one or more of the following functions: ester, amide, acid and/or hydroxyl.

Advantageously, the wax is chosen from fatty acids, fatty acid esters, fatty acid amides, hydrogenated oils, and mixtures thereof.

Advantageously, the fatty acids included in the composition of the wax are of plant origin.

Advantageously, the wax has a melting point Tm of greater than 70° C., preferably within the range from 70 to 160° C., preferably from 75 to 150° C., preferably from 80 to 140° C., preferably from 80 to 130° C., preferably from 80 to 120° C.

Advantageously, the wax includes at least 70% of ricinoleic acid, preferably at least 80%, preferably at least 90%, preferably at least 95% of ricinoleic acid.

The wax includes hydrogenated castor oil and/or hydroxystearamide monoethanolamine.

Advantageously, the particles have an apparent specific surface area within the range from 1 to 20 m2/g, preferably from 1 to 10 m2/g, preferably from 1 to 5 m2/g.

Advantageously, the powder according to the invention has an oil uptake, measured according to the standard NF ISO 787-5:1980 which is within the range from 0.3 to 0.9 g of oil/g of powder, preferably from 0.4 to 0.7 g of oil/g of powder, preferably from 0.4 to 0.6 g of oil/g of powder.

Advantageously, the powder contains at least one additive chosen from cosmetic raw materials, preferably of renewable origin.

Advantageously, the powder is entirely derived from renewable materials.

A subject of the present invention is also a process for manufacturing loose wax-based powder particles, said process comprising the following successive steps:
melting of the wax at a temperature above the melting point Tm of the wax, preferably at a temperature above Tm+10 to ensure that all of the material is molten;
atomization or spraying of the molten wax through a nozzle in a spraying tower;
cooling of the sprayed wax in less than 5 seconds so that the wax sets in the form of loose powder particles under the effect of a cooling carrier gas;
separation of the powder from the carrier gas, notably by a cyclone and/or a filter of suitable granulometry which allows the separation of the gas and the powder;
recovery of the loose wax powder.

Advantageously, the nozzle is designed to ensure the fluidity of the wax during its spraying in the form of microdroplets, said nozzle being a rotary nozzle or a twin-fluid nozzle, preferably a rotary nozzle.

Advantageously, the spraying step is performed in the presence of a co-injection of hot gas, such as hot air, with a temperature in the range from Tm to Tm+180° C., Tm being the melting point of the wax, preferably from Tm to Tm+175° C., preferably from Tm to Tm+150° C.

Advantageously, the spraying tower is fed with a cooling carrier gas with a temperature in the range from 10° C. to 50° C., preferably from 10 to 35° C., preferably from 15 to 25° C.

Advantageously, the loose wax powder according to the invention is manufactured by spray-cooling, cold spraying or cold atomization of said molten wax, preferably according to the spray-cooling process described above according to the invention.

A subject of the present invention is also the use of powder according to the invention in cosmetic, pharmaceutical or perfumery products.

For example, the powder may be used:
as a reinforcing and/or spreading agent in cosmetic formulations, notably those in stick form;
as a compacting agent in cosmetic formulations, notably those in pressed powder form;
as a mattifying agent; and/or
as a covering agent.

A subject of the present invention is also a cosmetic, pharmaceutical or perfumery composition, characterized in that it comprises powder particles as defined above.

Said composition is advantageously a colored, uncolored or transparent product chosen from the following products:
makeup products for the human face and body, such as foundation, tinted cream, loose or compact powder, eyeshadow, mascara, eyeliner, lipstick;
care products for the human face and body, such as cream, milk, lotion, mask, scrubbing product, cleansing and/or makeup-removing products, deodorants, antiperspirants, antiperspirants, shaving products, hair-removing products;
haircare products, such as shampoos, hairsetting products, hairstyle hold products, antidandruff products, products for preventing hair loss, products for combating dryness of the hair, hair dyes, bleaching products;
perfumery products, such as fragrance, milk, cream, loose or compact fragranced powder.

The powder of the invention has sebum-controlling properties, and also a mattifying effect. It is thus perfectly suitable for cosmetic products intended for making up and/or caring for human skin, in particular of the face, the neck and the body, and also pharmaceutical or perfumery products (fragrancing powder for the body or the feet, for example).

Advantageously, the powders of the invention afford, by virtue of their spherical shape, their particle size, their smooth surface and their apparent specific surface area, improved sensory properties and also improved reinforcing and compacting properties and improved spreading properties, when compared with the powders of the prior art.

### EXAMPLES

The examples that follow illustrate a preferred embodiment of the invention without, however, limiting it.

### Powders Tested

### According to the Invention

HCO test 1 or HCO 1: hydrogenated castor oil powder obtained by spray-cooling

HCO test 5 or HCO 5: hydrogenated castor oil powder obtained by spray-cooling

J100 test 5C: hydroxystearamide MEA powder obtained by spray-cooling

J100 test 6 L: hydroxystearamide MEA powder obtained by spray-cooling

### Not according to the Invention

Orgasol® Green Touch: Porous spheroidal PA 11 powder (8-15 microns) obtained by milling

Orgasol® 2002 EXD Nat Cos: Porous spheroidal PA 12 powder (10 microns) obtained directly by anionic polymerization

Milled Jagrowax-100 or milled J100 is hydroxystearamide MEA obtained by milling in an air jet mill.

Crayvallac PC nonspherical hydrogenated castor oil powder (10 microns), obtained by milling in an air jet mill

TABLE 1
Particle size distribution of the powders tested

| | Not according to the invention | | | | | | | | According to the invention | | | | | | | |
|---|---|---|---|---|---|---|---|---|---|---|---|---|---|---|---|---|
| | J100 | | Orgasol ® | | Orgasol ® | | | | J100 | | J100 | | | | | |
| | air jet | | Green | | 2002 EXD | | Crayvallac | | test | | test | | HCO | | HCO | |
| | mill | | Touch | | NAT COS | | PC | | 5C | | 6L | | test 1 | | test 5 | |
| DIO | 3.0 | μm | 9.9 | μm | 7.6 | μm | 3.9 | μm | 5.3 | μm | 5.2 | μm | 3.0 | μm | 6.4 | μm |
| D50 | 7.5 | μm | 14.5 | μm | 10.7 | μm | 11.6 | μm | 11.0 | μm | 10.5 | μm | 8.0 | μm | 14.8 | μm |
| D90 | 18.1 | μm | 19.4 | μm | 12.9 | μm | 24.2 | μm | 18.5 | μm | 18.3 | μm | 15.3 | μm | 26.5 | μm |

| | | | | | | | | |
|---|---|---|---|---|---|---|---|---|
| SPAN | 2 | 0.66 | 0.48 | 1.7 | 1.2 | 1.66 | 1.5 | 1.4 |
| <30 | 97.6% | 100% | 100% | 95.5% | 99.5% | 98.9% | 97.9% | 94.3% |
| micron | | | | | | | | |

Powders with a diameter substantially equal to 10 microns of PA 12 (not of renewable origin) and of PA 11 (of renewable origin) and of wax according to the invention or not according to the invention were evaluated in loose form by a trained sensory panel composed of 10 people

TABLE 2
Sensory analysis of the powders according to the invention: Study
conditions: 0.006 g of each powder (±0.0002 g)/deposition
of this amount on the forearm over an area S = 4.5 ×
2 (cm) Evaluation scale: from 0 (lowest score) to 5 (best score)
The Color criterion was evaluated between 0 (=white) and 5 (=yellow)

| | | | HCO test | HCO test |
|---|---|---|---|---|
| Powder tested: | J100 5c | J100 6L | 1 | 5 |
| Parameter tested | | | | |
| Color | 4 | 5 | 1 | 2 |
| Adhesion on uptake | 4 | 4 | 5 | 5 |
| Softness | 2 | 2 | 3 | 3 |
| Coverage | 2 | 2 | 3 | 3 |
| Mattness | 4 | 4 | 5 | 5 |
| Spreading | 1 | 1 | 1 | 2 |

TABLE 3
Sensory profile of the powders according to the invention
compared with the powders of the prior art: Evaluation
scale: from 0 (lowest score) to 100 (best score)

| | | | | | Orgasol ® | Orgasol ® |
|---|---|---|---|---|---|---|
| | J100 | J100 | HCO | HCO | 2002 EXD | Green |
| | 5C | 6L | test 1 | test 5 | NAT COS | Touch |
| Parameter | | | | | | |
| tested | | | | | | |
| Spreading | 5 | 4 | 2 | 30 | 23 | 33 |
| Coverage | 20 | 16 | 30 | 20 | 26 | 24 |
| Roughness | 15 | 25 | 18 | 15 | 2 | 18 |
| Creaminess | 35 | 40 | 30 | 10 | 29 | 10 |
| Velvet feel | 25 | 15 | 20 | 25 | 20 | 15 |

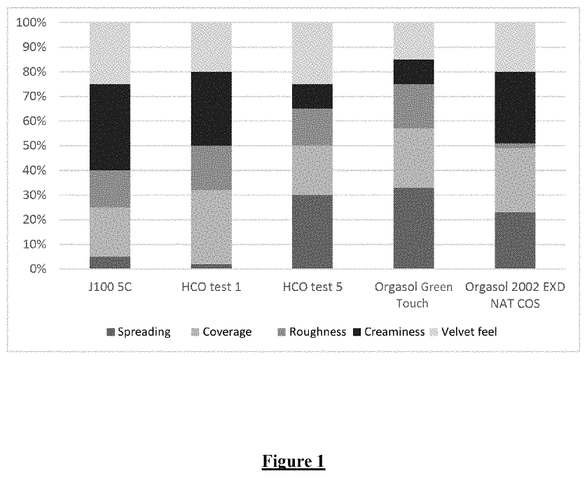

FIG. 1 represents the sensory profile of the powders according to the invention compared with that of the powders of the prior art.

TABLE 4
Oil uptake of the powders tested and of the powders of the prior art

| | Not according to the invention | | | | According to the invention | | | |
|---|---|---|---|---|---|---|---|---|
| | J100 | Orgasol ® | Orgasol ® | | J100 | J100 | | |
| | air jet | Green | 2002 EXD | Crayvallac | test | test | HCO | HCO |
| | mill | Touch | NAT COS | PC | 5C | 6L | test 1 | test 5 |
| Oil | — | 85 | 80 | 50 | 44 | 34 | 40 | 56 |
| uptake | | | | | | | | |
| (g/100 g) | | | | | | | | |

### Formulation 1: Rich Day Cream

A

Demineralized water (73% by mass)

Chlorphenesin in phenoxyethanol sold under the reference Microcare PHC by the company Thor (1.0% by mass)

Glycerol sold by the company Interchimie (3% by mass)

B1

Cetearyl Alcohol and Cetearyl Glucoside sold under the reference Montanov 68 by the company SEPPIC (5.0% by mass)

Butyrospermum parkii butter sold under the reference Lipex Shea by the company

Unipex (3.0% by mass)

B2

Caprylic/capric triglycerides sold by the company Interchimie (10.0% by mass)

C

Sodium Acrylate/Sodium Acryloyldimethyl Taurate Copolymer & Isohexadecane & Polysorbate 80 sold under the reference Simulgel EG by the company SEPPIC (1.0% by mass)

D

Demineralized water (1 to 4% by mass)

D2

HCO or Jagrowax sensory powder (1 to 3% by mass)

### Procedure

The ingredients of phase A are weighed out and added successively with stirring at 250 rpm at room temperature. The mixture is stirred for 1 hour to obtain a fluid texture and is then heated to 75° C. Phase B1 is heated to 75° C. and then added to phase A with stirring (250 rpm).

The mixture is stirred with an Ultra-Turrax blender (2510 rpm) so as to introduce phase B2 over a few minutes. After the introduction, the solution is returned to gentle stirring (300 rpm) and the temperature is reduced to 35° C. Phase C is added with vigorous stirring at 1280 rpm. The mixture is then placed at room temperature for the addition of phase D1, followed by D2 until the formulation is homogeneous.

### Result

The day cream containing the experimental powders of HCO (or of Jagrowax J100) applies very well and has good glidance.

The result is uniform and matt, with no powdery effect. The feel is soft and not tacky.

TABLE 5
Analysis of the sensory properties of the rich day cream (emulsion):

| | | | | | | | | HCO | HCO | HCO | HCO | HCO | HCO |
|---|---|---|---|---|---|---|---|---|---|---|---|---|---|
| | | | | | J100 | J100 | J100 | test | test | test | test | test | test |
| | Placebo: | 2002 | Green | Crayvallac | (5C) - | (5C) - | (5C) - | 1 - | 1 - | 1 - | 5 - | 5 - | 5 - |
| | Water | EXD | Touch | PC | 1% | 2% | 3% | 1% | 2% | 3% | 1% | 2% | 3% |
| Gloss | 8 | 4 | 5 | 5 | 6 | 5 | 4 | 6 | 5 | 4 | 6 | 5 | 6 |
| Spreadability | 8 | 8 | 8 | 8 | 5 | 5 | 4 | 7 | 6 | 6 | 7 | 6 | 5 |
| Thickness/ | 3 | 5 | 5 | 4 | 6 | 6 | 7 | 5 | 6 | 7 | 5 | 5 | 5 |
| density | | | | | | | | | | | | | |
| Tackiness | 7 | 4 | 4 | 5 | 6 | 5 | 4 | 5 | 4 | 4 | 5 | 4 | 4 |
| Speed of | 3 | 7 | 7 | 7 | 4 | 5 | 6 | 6 | 7 | 7 | 5 | 5 | 5 |
| penetration | | | | | | | | | | | | | |

Compared with the placebo (without powder, percentage replaced simply with water), the introduction of Jagrowax J100 (5C) according to the invention into the rich day cream brings about a strong decrease in the gloss. The gloss effect decreases when the Jagrowax concentration increases and gives rise to a matt effect.

Just like the Orgasol® multifunctional powders, the introduction of Jagrowax brings about a slight increase in the density of the cream and thus improvement of the texture.

The tackiness is also reduced proportionately to the addition of Jagrowax to the formulation on account of its oil-absorbing properties.

The speed of penetration is also increased with the proportion of Jagrowax and brings about a decrease in the tackiness.

Similarly, for the HCO powder with a diameter D50v of 15 μm (HCO test 5):
increase in the speed of penetration relative to the placebo
decrease in the tackiness
slight increase in the texture/density
spreadability
decrease in the gloss

Similarly, for the HCO powder of small diameter (HCO test 1—of diameter D50v 8 μm):

Sensory profile similar to that of Green Touch for 3%. Only the texture is different and a slight decrease in the spreadability is observed.

### Formulation 2: Lipstick

A
Octyldodecanol sold under the reference DUB ODOL by the company Stearinerie Dubois (18.1% by mass)
Candelilla wax sold under the reference Cerilla Raffinée Paillettes by the company Univar (11.1% by mass)
Carnauba wax sold under the reference Cerauba T1 by the company Univar (1.5% by mass)
White beeswax sold under the reference Cerabeil Blanche by the company Univar (7.6% by mass)
Caprylic/capric triglycerides sold under the reference DUB MCT by the company Stearinerie Dubois (3.5% by mass)
Isostearyl isostearate sold under the reference DUB ISIS by the company Stearinerie Dubois (9.0% by mass)
Pentaerythrityl tetraisostearate sold under the reference DUB PTIS by the company Stearinerie Dubois (30.7% by mass)
VP/Hexadecene copolymer sold under the reference Antaron V216 by the company Stearinerie Dubois (4.0% by mass)

B
Experimental powder of HCO or of Jagrowax J100 (Arkema)—3% by mass

C
Iron oxide and mica pigment sold under the reference Colorona Bordeaux by the company Merck (3% by mass)

### Procedure

The constituents of phase A are weighed out and then placed on a water bath (90° C.) with stirring. When the mixture is homogeneous, the Arkema sensory powder is added with stirring (410 rpm). After the addition, stirring is maintained for 15 minutes. Phase C is then added with stirring (580 rpm, 10 min). The lipstick is maintained at 90° C. and then poured into a mold heated beforehand at 42° C. for 2 hours. After having been cast, the mold is stored for 15 minutes at room temperature and then for 1 hour at −18° C.

### Result

### Sensory Properties:

The lipstick containing the experimental powders of HCO (or of Jagrowax J100) applies very well and has good glidance.

The result is uniform and matt, the color is homogeneous. The feel is soft and not tacky.

TABLE 6
criteria for evaluating the sensory powders in the lipsticks

| Criterion | Definition | Evaluation technique |
|---|---|---|
| Long-lasting | Ability of the lipstick | Apply the lipstick in a line of 5 cm |
| effect | to resist over time | to the forearm, applying a constant |
| | | pressure. |
| | | Make three return trips. |
| | | Pass a handkerchief over and see |
| | | whether or not the color has |
| | | diminished. |
| Application | Ease of application and | Apply the lipstick in a line of 5 cm |
| | spreadability of the | to the forearm, applying a constant |
| | lipstick | pressure. |
| | | Make three return trips. |
| | | Evaluate the ease or difficulty of |
| | | applying the lipstick. |
| Fondant nature/ | The product melts on | Apply the lipstick in a line of 5 cm |
| creaminess | contact with the lips | to the forearm, applying a constant |
| | and changes in | pressure. |
| | viscosity during | Make three return trips. |
| | application. | Evaluate the fondant nature, |
| | | noting any change in texture. |
| Coverage | The product does not | Apply a pen mark to the forearm |
| | allow the applied | and then apply the lipstick in a line |
| | surface to show | of 5 cm, applying a constant |
| | through. | pressure. |
| | | Make three return trips. |
| | | See if the pen mark is visible. |
| | | Evaluate the residual intensity of |
| | | the mark. |
| Tackiness | The product sticks to | Apply the lipstick in a line of 5 cm |
| | the lips after | to the forearm, applying a constant |
| | application. | pressure. |
| | | Make three return trips. |
| | | Press the index finger once on the |
| | | application. |
| | | Evaluate the tackiness on |
| | | removing the finger. |
| Color | Lipstick deposit which | Apply the lipstick in a line of 5 cm |
| homogeneity | covers the lips | to the forearm, applying a constant |
| | uniformly. | pressure. |
| | | Make three return trips. |
| | | Evaluate whether or not the |
| | | deposit is uniform. |

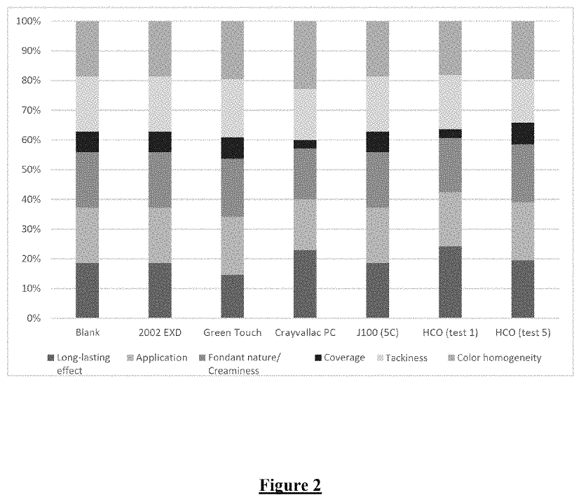

FIG. 2 shows the sensory evaluation of the sensory powders in the lipstick formulations.

TABLE 7
Sensory evaluation of the sensory powders in the lipstick formulations.

| | | Orgasol ® | Orgasol ® | | | | |
|---|---|---|---|---|---|---|---|
| | | 2002 EXD | Green | Crayvallac | J100 | HCO | HCO |
| | Placebo* | NAT COS | Touch | PC | (5C) | (test 1) | (test 5) |
| Properties | | | | | | | |
| Long-lasting | 3 | 4 | 3 | 4 | 4 | 4 | 4 |
| effect | | | | | | | |
| Application | 3 | 4 | 4 | 3 | 4 | 3 | 4 |
| Fondant | 3 | 4 | 4 | 3 | 4 | 3 | 4 |
| nature/ | | | | | | | |
| creaminess | | | | | | | |
| Coverage | 1 | 2 | 2 | 0.5 | 1.5 | 0.5 | 1.5 |
| Tackiness | 4 | 4 | 4 | 3 | 4 | 3 | 3 |
| Color | 3 | 4 | 4 | 4 | 4 | 3 | 4 |
| homogeneity | | | | | | | |

*the placebo contains 3% of octyldodecanol, sold under the reference DUB ODOL by the company Stearinerie Dubois

### Breaking Strength Test of the Lipstick Stalk:

The lipstick is held horizontally by clamps, with the stalk fully extended out. The handle of an empty bucket is placed 0.5 mm from the base of the stalk. The bucket is filled with water continuously until the stalk breaks. The final amount of water added is weighed. The test is repeated five times and the average value is retained.

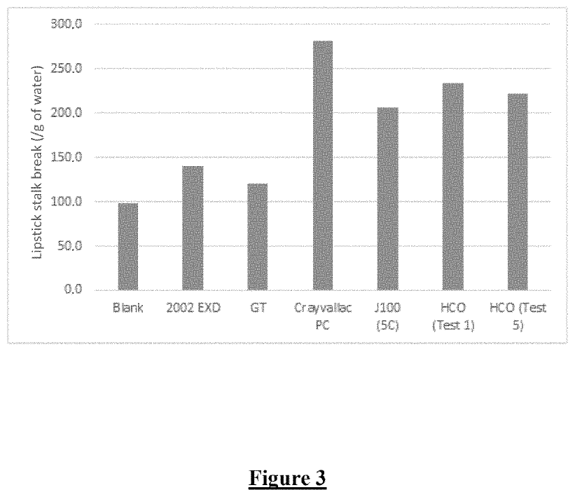

The graph of FIG. 3 demonstrates better resistance of the lipstick stalk for the powders according to the invention (HCO test 1 and HCO test 5) compared with the blank and also with the Orgasol® Green Touch and Orgasol® 2002 EXD NAT COS powders. The nonspherical Crayvallac PC powder, obtained by milling with an air jet mill, also has a high breaking strength, but this value proves to be too high since it prevents good spreading of the lipstick because the lipstick stalk is too compact, and the color is not homogeneous. In the latter case, it is observed that the lipstick is difficult to apply, or even is impossible to apply.

The sensory properties obtained for the HCO test 1 and 5 and Jagrowax J100 5C and 6 L powders according to the invention are similar to those for Orgasol® Green Touch, i.e. homogeneity of the color and a long-lasting effect.

The powders according to the invention show excellent reinforcing properties, which are visible via improvement of the breaking strength of the lipstick stalk.

### Formulation 3: Pressed Powder

A
Talc, sold under the reference Emprove Parteck LUB Talc by the company Merck (46.2% by mass)
Mica, sold under the reference Mica Concord Grade 700 by the company Sciama (30% by mass)
Salicylic acid, sold under the reference Salicylic Acid Pharma by the company Cooper Industries (0.2% by mass)
Experimental powder of HCO or of Jagrowax J100 (Arkema)—10% by mass
Pigments, sold under the reference Unipure by the company Sensient (yellow 0.6% by mass, read 0.4% by mass, black 0.1% by mass and white 3% by mass)

B
Octyldodecanol and octyldodecyl xyloside, sold under the reference Fluidanov 20× by the company SEPPIC (5.5% by mass)
Isostearyl isostearate, sold under the reference Crodamol ISIS by the company Croda (4% by mass)

### Procedure

The constituents of phase A are weighed out and then mixed until homogeneous. When the mixture is homogeneous, the Arkema sensory powder is added and then mixed. Phase B is then added to the surface of the powder and mixed (3×5 s). The powder obtained is screened and compacted.

### Result

Sensory test of the pressed powders

Study conditions: uptake by five rotations on pressed powder/deposition of this amount onto the forearm S=4.5×2 cm

Evaluation scale: from 0 (lowest score) to 5 (highest score)

TABLE 8
Sensory profile of the powders in the pressed
powder formulations according to the invention
compared with the powders of the prior art

| | | Orgasol ® 2002 | HCO | HCO |
|---|---|---|---|---|
| | Placebo* | EXD NAT COS | (test 1) | (test 5) |
| Properties | | | | |
| Color | 3.5 | 3 | 3 | 3 |
| Adhesion on uptake | 5 | 2.5 | 3 | 3 |
| Homogeneity after | 2.5 | 4.5 | 2 | 2 |
| spreading | | | | |
| Softness | 3.5 | 4.5 | 2 | 2 |
| Coverage | 3 | 2 | 2.5 | 3.5 |
| Mattness | 4 | 3.5 | 4.5 | 4.5 |

*The placebo contains 10% of talc, sold under the reference Emprove Parteck LUB Talc by the company Merck (56.2%).

The pressed powders containing the HCO experimental powders apply well. The result is uniform, matt and covering.

### Drop Test

The protocol which was followed to define the drop strength of the compact powders consists in
standing over a metal plate and holding a 30 cm graduated ruler vertically in one hand,
holding the pot horizontally in the other hand—the bottom of the pot facing downwards—over the plate,
releasing the pot and repeating the operation until the first crack or break appears, and recording the number of drops performed,
repeating the operation on at least two or three pots, and then taking an average of the results.

The results show that the pressed powders containing the HCO experimental powders have much better compaction than the powders of the prior art.

TABLE 9
drop tests of the powders in the pressed powder formulations according
to the invention compared with the powders of the prior art

| | Number of drops before breaking |
|---|---|
| Placebo | 35 |
| Orgasol ® 2002 EXD NAT COS | 38 |
| HCO (test 1) | 70 |
| HCO (test 5) | 72 |

In summary, the process for manufacturing the powder of the invention makes it possible to dispense with the consumption of oil, to reduce the energy consumption and to make use of raw materials obtained from plant crops. It also has a lower manufacturing cost and a favorable energy balance.

1. A loose wax powder obtained from renewable materials, wherein particles of said loose wax powder have a particle size such that:
the D90v is less than 20 micron
the (D90-D10)/D50 result is less than 1.8
the volume percentage of particles with a diameter of at least 30 micron is less than 5%, and
wherein the wax is selected from the group consisting of fatty acids, fatty acid esters, fatty acid amides, hydrogenated oils, and mixtures thereof.

2. The powder as claimed in claim 1, wherein the volume-median diameter D50v is within the range from 2 to 20 microns.

3. The powder as claimed in claim 1, wherein the SPAN is less than 1.5.

4. The powder as claimed in claim 1, wherein the content of particles with a diameter of at least 30 micron is less than 3%, preferably less than 2%, preferably less than 1%.

5. The powder as claimed in claim 1, wherein the particles are of substantially spherical shape.

6. The powder as claimed in claim 5, wherein said particles have a sphericity index Is within the range from 1 to 1.2, the Is being measured by the ratio between the largest diameter of the particle to the smallest diameter of the particle, the diameters being measured by SEM.

7. The powder as claimed in claim 1, wherein the surface of the wax particles is smooth.

8. The powder as claimed in claim 1, wherein the fatty acids included in the composition of the wax are of plant origin.

9. The powder as claimed in claim 1, wherein the wax has a melting point Tm of greater than 70° C.

10. The powder as claimed claim 1, wherein the wax comprises at least 70% of ricinoleic acid.

11. The powder as claimed in claim 1, wherein the wax includes hydrogenated castor oil and/or hydroxystearamide monoethanolamine.

12. The powder as claimed in claim 1, wherein the particles have an apparent specific surface area within the range from 1 to 20 m2/g.

13. The powder as claimed in claim 1, wherein the oil uptake of said powder, measured according to the standard NF ISO 787-5:1980 is within the range from 0.3 to 0.9 g of oil/g of powder.

14. The powder as claimed in claim 1, wherein it contains at least one additive chosen from cosmetic raw materials, preferably of renewable origin.

15. The powder as claimed in claim 1, wherein it is totally derived from renewable materials.

16. A process for manufacturing wax-based loose powder particles as defined in claim 1, said process comprising the following successive steps:
melting a wax at a temperature above the melting point Tm of the wax, to ensure that all of the material is molten;
spraying the molten wax through a nozzle in a spraying tower;
cooling the sprayed wax in less than 5 seconds so that the wax sets in the form of loose powder particles under the effect of a cooling carrier gas;
separating the powder from the carrier gas;
recovering the loose wax powder.

17. The process as claimed in claim 16, in which the nozzle is a rotary nozzle or a twin-fluid nozzle.

18. The process as claimed in claim 16, wherein the spraying step is performed in the presence of a co-injection of hot gas with a temperature in the range from Tm to Tm+180° C., Tm being the melting point of the wax.

19. The process as claimed in claim 16, in which the spraying tower is fed with a cooling carrier gas with a temperature in the range from 10° C. to 50° C., preferably from 10 to 35° C., preferably from 15 to 25° C.

20. The process as claimed in claim 16, wherein said cooling comprises spray-cooling, cold spraying or cold atomization of said molten wax.

21. (canceled)

22. (canceled)

23. The cosmetic, pharmaceutical or perfumery composition of claim 27, wherein said composition is a reinforcing and/or spreading agent in cosmetic formulations, in stick form.

24. The cosmetic, pharmaceutical or perfumery composition of claim 27, wherein said composition is a compacting agent in cosmetic formulations, in pressed powder form.

25. The cosmetic, pharmaceutical or perfumery composition of claim 27, wherein said composition is a mattifying agent.

26. The cosmetic, pharmaceutical or perfumery composition of claim 27, wherein said composition is a covering agent.

27. A cosmetic, pharmaceutical or perfumery composition, wherein said composition comprises powder particles as defined in claim 1.

28. The composition as claimed in claim 27, said composition being a colored, uncolored or transparent product selected from the group of products consisting of:
makeup products for the human face and body, such as foundation, tinted cream, loose or compact powder, eyeshadow, mascara, eyeliner, lipstick;
care products for the human face and body, cream, milk, lotion, mask, scrubbing product, cleansing and/or makeup-removing products, deodorants, antiperspirants, antiperspirants, shaving products, hair-removing products;
haircare products, shampoos, hairsetting products, hairstyle hold products, antidandruff products, products for preventing hair loss, products for combating dryness of the hair, hair dyes, bleaching products;
perfumery products, fragrance, milk, cream, and loose or compact fragranced powder.
